(19) United States
(12) Patent Application Publication
(10) Pub. No.: US 2005/0271738 A1
(43) Pub. Date: Dec. 8, 2005

(54) Methods and compositions for treating tissue defects using pulsed electromagnetic field stimulus

(75) Inventor: Bruce J. Simon, Mountain Lake, NJ (US)

Correspondence Address: David L Suter;Harness Dickey & Pierce, PO Box 828, Bloomfield Hills, MI, 48303, US

(73) Assignee: EBI, L.P., PARSIPPANY, NJ (US)

(21) Appl. No.: 10/522,351

(22) PCT Filed: Jul. 24, 2003
(86) PCT No.: PCT/US03/23146

§ 371 Date: Jan. 26, 2005

Related U.S. Application Data
(60) Provisional application No. 60/398,980, filed on Jul. 26, 2002.

(51) Int. Cl. A61K 35/12

(52) U.S. Cl. 424/520

(57) ABSTRACT

Methods of enhancing cell proliferation in tissue cultures and at the site of tissue defects in human or other animal subjects, comprising the steps of: (a) culturing a living tissue in a medium to form a tissue culture; (b) subjecting said tissue culture to an electromagnetic field; (c) extracting said medium from said tissue culture; and (d) administering said medium to the site of said tissue defect. Preferably, the tissue culture comprises endothelial cells. The present invention also provides compositions for the treatment of tissue defects in a human or other animal subject, comprising a safe and effective amount of a medium produced by electromagnetic stimulation of a tissue culture. Preferably, the compositions comprise a pharmaceutically acceptable carrier, such as hyaluronic acid, gelatin, collagen, cellulose ether, and osteoconductive carriers.

### FIELD OF THE INVENTION

The present invention relates generally to compositions and methods of treating tissue defects in human or other animal subjects. More particularly, the present invention relates to such methods, and compositions made using such methods, using electromagnetic fields to stimulate tissue cell cultures.

### BACKGROUND OF THE INVENTION

The formation of blood vessels from pre-existing endothelial cells (angiogenesis) is an integral component of physiologic processes such as growth and repair as well as pathologic states such as various malignancies. The importance of new tissue growth is also clear in ischemic conditions that result from inadequate angiogenesis. As a result, there has been extensive research to develop potential methods to stimulate angiogenesis, known as “therapeutic angiogenesis.”

To date, most research in the area of therapeutic angiogenesis has focused on the delivery of pro-angiogenic growth factors, either as recombinant proteins or via gene delivery. For instance, vascular endothelial growth factor (VEGF) and fibroblast growth factor (FGF) gene transfer have produced promising results in treating vascular diseases such as myocardial ischemia and peripheral vascular disease. Furthermore, the delivery of growth factors for the stimulation of bone tissue and cartilage have produced promising results. Such growth factors include TGF-β, PDGF, IGF-I, IGF-II, FGF, EGF, and VEGF.

Recently, researchers have turned their attention to cell-based approaches rather than the administration of cytokines, such as the delivery of bone-marrow stem cells or related cell types to induce neovascularization. Such techniques are disclosed in Kamihata, H. et al., “Implantation of bone marrow mononuclear cells into ischemic myocardium enhances collateral perfusion and regional function via side supply of angioblasts, angiogenic ligands, and cytokines,” Circulation 104, 1046-1052 (2001); Kalka, C. et al., “Transplantation of ex vivo expanded endothelial progenitor cells for therapeutic neovascularization,” Proc Natl Acad Sci USA 97, 3422-3427 (2000); and Carmeliet, P. & Luttun, A., “The emerging role of the bone marrow-derived stem cells in (therapeutic) angiogenesis,” Thromb Haemost 86, 289-29 (2001). Both approaches, however, exhibit potential limiting factors such as immunogenicity, targeting specificity, maintenance of therapeutic levels, and invasiveness.

### SUMMARY OF THE INVENTION

The present invention provides a method of enhancing cell proliferation in tissue cultures and at the site of tissue defects in human or other animal subjects. In one embodiment, the present invention provides a method of treating a tissue defect in a human or other animal subject, comprising the steps of:
(a) culturing a living tissue in a medium to form a tissue culture;
(b) subjecting said tissue culture to an electromagnetic field;
(c) extracting said medium from said tissue culture; and
(d) administering said medium to the site of said tissue defect.
   Preferably, the tissue culture comprises endothelial cells in one embodiment, the tissue culture comprises autologous tissues from the subject to whom the medium is administered. In another embodiment, the present invention provides methods of enhancing cell proliferation in a tissue culture of interest, comprising the steps of:
(a) culturing a living tissue in a medium to form a first tissue culture;
(b) subjecting said first tissue culture to an electromagnetic field;
(c) extracting said medium from said first tissue culture; and
(d) administering said medium to said tissue culture of interest.

In another embodiment, the present invention provides compositions for the treatment of tissue defects in a human or other animal subject, comprising a safe and effective amount of a medium produced by electromagnetic stimulation of a tissue culture. In one embodiment, the tissue culture comprises autologous tissues from the subject to whom the medium is administered. The invention also provides compositions for generating new tissue growth comprising a soluble factor or stimulating factor secreted in a culture by living tissue cells following electromagnetic field stimulation. Preferably, the compositions comprise a pharmaceutically-acceptable carrier, such as hyaluronic acid, gelatin, collagen, cellulose ether, and osteoconductive carriers.

It has been found that the compositions and methods of this invention afford benefits over compositions and methods among those known in the art. Such benefits include one or more of enhanced efficacy, reduced side effects, ease of administration, and reduced cost of therapy. Specific benefits and embodiments of the present invention are apparent from the detailed description set forth herein. It should be understood that the detailed description and specific examples, while indicating the preferred embodiment of the invention, are intended for purposes of illustration only and are not intended to limit the scope of the invention.

### DESCRIPTION

The present invention involves the treatment of tissue defects in humans or other animal subjects. Specific materials to be used in the invention must, accordingly, be pharmaceutically acceptable. As used herein, such a “pharmaceutically acceptable” component is one that is suitable for use with humans and/or animals without undue adverse side effects (such as toxicity, irritation, and allergic response) commensurate with a reasonable benefit/risk ratio.

Tissue Defects

The compositions and methods of this invention may be used to repair various tissue defects. As referred to herein such “tissue defects” include any condition involving tissue which is inadequate for physiological or cosmetic purposes. Such defects include those that are congenital, the result from disease or trauma, and consequent to surgical or other medical procedures. Embodiments include treatment for vascular, bone, skin, and organ tissue defects. Such defects include those resulting from osteoporosis, spinal fixation procedures, hip and other joint replacement procedures, chronic wounds, myocardial infarction, fractures, sclerosis of tissues and muscles, Alzheimer's disease, and Parkinson's disease. (As used herein, the word “include,” and its variants, is intended to be non-limiting, such that recitation of items in a list is not to the exclusion of other like items that may also be useful in the materials, compositions, devices, and methods of this invention.)

In one embodiment, the compositions and methods of this invention may be used to repair bone or cartilage defects. A preferred embodiment is for the treatment of bone defects. As referred to herein such “bone defects” include any condition involving skeletal tissue which is inadequate for physiological or cosmetic purposes. Such defects include those that are congenital, the result from disease or trauma, and consequent to surgical or other medical procedures. Such defects include those resulting from bone fractures, osteoporosis, spinal fixation procedures, hip and other joint replacement procedures. (As used herein, the words “preferred” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.)

In one embodiment, the methods of this invention are for therapeutic angiogenesis of a human or animal subject. Such methods are those which enhance cell proliferation in tissue, preferably which is at or near the site of a defect, so as to cause repair or enhancement of tissue. Preferably such methods enhance growth of blood vessels. Preferably such methods enhance tubulization. Without being bound by theory, in some embodiments methods of the present invention employ a calcium-dependent secondary messenger pathway.

Culturing of Tissue

The methods of the present invention comprise culturing of a living tissue. Such tissues include vascular, skin, skeletal, and other organ tissues. A preferred embodiment comprises the use of vascular tissues. Another embodiment comprises the use of skeletal tissue. Such tissues may be from human or other animal sources, preferably from human. The tissue used in the current invention may be obtained by appropriate biopsy or upon autopsy. In one embodiment, the tissue is autologous, obtained from the identical subject to whom the medium is administered in the methods of this invention. In another embodiment, the tissue is allogenic, obtained from a different subject of the same species. In another embodiment, the tissue is xenogenic, obtained from a subject of a different species. In one embodiment, the cells are stromal cells. Stromal cells useful herein include endothelial cells, pericytes, macrophages, monocytes, leukocytes, plasma cells, mast cells, keratinocytes, chondrocytes or adipocytes. The stromal cells may also comprise fibroblasts with or without additional cells and/or other elements. Preferably the stromal cells are endothelial cells.

The endothelial cells are cultured within a medium, such as endothelial cell basal medium (EBM-2), and supplemented with various growth factors and other extracellular matrix proteins and culturing solutions. Such growth factors include but are not limited to VEGF-1, fibroblast growth factor-2 (FGF-2), epidermal growth factor (EGF), insulin-like growth factor-1 (IGF-1), TGF-β, PDGF, IGF-I, IFG-II and EGM. An acid, such as ascorbic acid, may be added to further provide optimal culturing conditions.

Stromal cells comprising endothelial cells derived from adult or fetal tissue, with or without other cells and elements described below, are cultured in a medium. These endothelial cells may be derived from organs, such as skin, liver, pancreas, etc. which can be obtained by biopsy, where appropriate, or upon autopsy. In one embodiment, endothelial cells are obtained from the human umbilical vein. Endothelial cells may be readily isolated by disaggregating an appropriate organ or tissue which is to serve as the source of the cells. This can be readily accomplished using techniques including those known in the art. For example, the tissue or organ can be disaggregated mechanically and/or treated with digestive enzymes and/or chelating agents that weaken the connections between neighboring cells making it possible to disperse the tissue into a suspension of individual cells without appreciable cell breakage. Enzymatic dissociation can be accomplished by mincing the tissue and treating the minced tissue with any of a number of digestive enzymes either alone or in combination. Such enzymes include but are not limited to trypsin, chymotrypsin, collagenase, elastase, hyaluronidase, DNase, pronase, dispase, and mixtures thereof. Mechanical disruption can also be accomplished by a number of methods including the use of grinders, blenders, sieves, homogenizers, pressure cells, and insonators.

Once the tissue has been reduced to a suspension of individual cells, the suspension can be fractionated into subpopulations from which the fibroblasts and/or other stromal cells and/or elements can be obtained. This also may be accomplished using standard techniques for cell separation including, but not limited to, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counter-streaming centrifugation), unit gravity separation, countercurrent distribution, electrophoresis and fluorescence-activated cell sorting. Other cells may be isolated, such cells include fibroblasts, pericytes, macrophages, monocytes, plasma cells, mast cells, adipocytes, keratinocytes, and chondrocytes. These stromal cells can be readily derived from appropriate organs such as skin, liver, etc., using methods known, such as those discussed above.

In another embodiment, microcarriers are provided, such as Cytodex 3®, and suspended in solution with the endothelial cells of the culture prior to electromagnetic field exposure. A fibrin gel is further provided to serve as a matrix. The use of microcarriers enables cells to sprout from a single foci, and the fibrin gel provides the matrix necessary for such sprouting. In another embodiment, a 3-dimensional angiogenesis assay is used to develop the electromagnetic field stimulated cell cultures.

Electromagnetic Field Stimulation

The medium of the cell culture is subjected to electromagnetic field stimulation. Preferably the electromagnetic field stimulation is pulsed, although other forms of radiation sources can be used. Preferably the cell culture is exposed to electromagnetic field stimulation during the incubation period of the cell culture. Preferably the culture is subjected to electromagnetic radiation for at least about 30 minutes, preferably at least about 1 hour, preferably at least about 8 hours, preferably at least about 20 hours. Various devices may be used to expose the cell culture to electromagnetic fields. Preferred devices include those wherein the electromagnetic field source may be placed inside a standard incubator, while the power source remains outside of the incubator. Equipment useful in the methods of this invention includes equipment comprising Helmholz coils, supplied by EBI, L. P. (Parsippany, N.J., U.S.A.). Depending on the type of tissue cells being cultured, the pulse frequency and strength of the electromagnetic field radiation required, as well as the position of the culture within the electromagnetic field, can vary.

Administration of Medium

The methods of this invention comprise administration of the medium in which tissue has been cultured. As referred to herein, the medium is a portion of the tissue culture that does not comprise significant quantities of viable cells. Preferably, the medium does not comprise any living cells. In one embodiment, cells have been substantially removed from the medium by filtration or other methods among those known in the art. In one embodiment, the medium is sterilized prior to administration to a subject, using radiation or other methods which do not denature growth factors or other proteins that may be present in the medium.

Compositions

The present invention provides compositions comprising tissue cell culture medium exposed to electromagnetic field stimulus. In one embodiment, the composition consists essentially of the medium. In another embodiment, the composition comprises the medium and a pharmaceutically-acceptable carrier. In a preferred embodiment, the composition is lyophilized or otherwise processed to form a dry powder which may be administered directly to the site of the tissue or bone defect, or mixed with saline or another suitable carrier prior to administration.

Preferred pharmaceutically acceptable carriers include saline, hyaluronic acid, cellulose ethers (such as carboxymethyl cellulose), collagen, gelatin, an osteoconductive carrier, and mixtures thereof. Osteoconductive carriers include allograft bone particles, demineralized bone matrix, calcium phosphate, calcium sulfate, hydroxyapatite, polylactic acid, polyglycolic acid and mixtures thereof. The compositions may optionally comprise other tissue/bone growth active materials, such as other growth factors, hormones (e.g., estrogen, calcitonin, parathyroid hormone, selective estrogen receptor modulators), and phosphonates (e.g., bisphosphonates).

The compositions of the present invention may be made in any of a variety of ways. In one embodiment, the matrix or medium is reduced to a powder, and the powder is coated on, or otherwise mixed with, a carrier. In another embodiment, the matrix or medium is mixed with the carrier, and the mixture is lyophilized.

In a preferred embodiment, the carrier comprises an osteoconductive carrier selected from the group consisting of calcium phosphate, hydroxyapatite, calcium sulfate, and mixtures thereof, preferably as a hardening paste. In one embodiment, the electromagnetically stimulated culture medium is mixed with the carrier during formation of the hardening paste, and the paste applied to the site of the bone defect. In another embodiment, the electromagnetically stimulated culture medium is mixed with the carrier during formation of the hardening paste, the paste allowed to harden, and then the paste is broken up into small particles before administration to the site of the bone defect.

The following non-limiting examples illustrate the compositions and methods of the present invention.

### EXAMPLE 1

Human umbilical vein endothelial cells (HUVECs), such as those commercially available from Cambrex Corporation (East Rutherford, N.J., U.S.A.) are cultured in endothelial cell basal medium (EBM-2) supplemented with EGM-2MV (microvascular endothelial growth medium, sold by Cambrex Corporation) single aliquots consisting of 5% FBS, VEGF-1, fibroblast growth factor-2 (FGF-2), epidermal growth factor (EGF), insulin-like growth factor-1 (IGF-1), and absorbic acid. Cells are grown until they reach confluence (passages 4-7), at which time they are harvested with trypsin-EDTA.

The tissue culture is subjected to pulsed EMF using commercially available equipment comprising Helmholz coils, supplied by EBI, L. P. (Parsippany, N.J., U.S.A.). EMF is delivered at 4.5 second pulses and a uniform magnetic field of 15 Hertz. Cultures are placed in the middle of EMF incubator.

The medium is separated from the cells in the culture, and admixed with collagen to form a composition of this invention. The composition is administered to the site of a skin wound in a human subject, accelerating healing.

### EXAMPLE 2

Microcarriers (MCs) (Cytodex™ 3, sold by Amersham Biosciences, AB, Uppsala, Sweden) are prepared by suspending MC powder in phosphate buffered saline (PBS), at a concentration of 1 gram MC/100 ml PBS. MCs are autoclaved and resuspended in 15 ml of EGM-2MV. HUVECS are added to 2 ml of MC solution and incubated for 4 hours at 37° C. and 5% CO₂. EGM-2MV is then added to achieve a final total volume of 10 mL, and the mixture incubated for 2-4 days until the cells become confluent on the MCs.

Fibrin gels are prepared as disclosed in Nehls, V. & Drenckhahn, D., “A novel, microcarrier-based in vitro assay for rapid and reliable quantification of three-dimensional cell migration and angiogenesis.” Microvasc Res 50, 311-322, (1995). Fibrinogen (Sigma-Aldrich, Corporation, St. Louis, Mo., U.S.A.) is dissolved in PBS at a concentration of 2.5 mg/ml, and 200 U/ml of aprotinin is added to prevent excessive fibrinolysis. The solution is filtered (0.2 um) and 1.5 ml is added to each well of a 6 well-plate. HUVEC-seeded MCs are added to each well and swirled to allow even distribution. Polymerization of the gel is induced by the addition of 0.625 U/ml (12 ul) of thrombin. Complete polymerization occurs approximately 30 minute following incubation, at which time 2 ml of EGM-2MV media are added to each well. Fibrin gels with HUVEC-seeded MCs are cultured in the presence of pulsed EMF using commercially available equipment comprising Helmholz coils, supplied by EBI, L. P. (Parsippany, N.J., U.S.A.). EMF is delivered at 4.5 second pulses and a uniform magnetic field of 15 Hertz. Cultures are placed in the middle of EMF incubator, and EMF is applied for 7-10 days, 8 hours/day. The degree of angiogenesis is quantified by choosing 50 MCs at random and counting the number of MCs with tubules greater than one MC diameter or twice the MC diameter.

The medium is separated from the cells in the culture, and admixed with demineralized bone matrix and gelatin to form a paste composition of this invention. The composition is administered to the site of a bone fracture in a human subject, accelerating healing.

### EXAMPLE 3

HUVECs are prepared for the proliferation assays as described above. After a 24 hour starvation period, the HUVECs are grown in media obtained from HUVEC cultures that had been incubating in pulsed EMF for 24 hours, as described above. Following an additional 24 hours, the media is again replaced with a fresh sample of EMF-cultured media. At the completion of 48 hours of incubation under normal conditions, a thymidine proliferation assay is performed. Three hours prior to the completion of the assay 5 μL of radioactive thymidine is added to each well. The cells are allowed to proliferate for an additional 3 hours, at which time, they are washed with PBS ×3, followed by 10% trichloroacetic acid (TCA) ×3. After the final wash, 2 mL of 1N NaOH is added to each well, incubated for 30 minutes, and neutralized by 2 mL 1N HCl. Finally, each sample is evaluated by a scintillation counter to correlate cell proliferation with the amount of radioactivity. The test demonstrates an enhancement in cell proliferation versus similar cultures that have been grown without exposure to the EMF-treated medium.

The examples and other embodiments described herein are exemplary and not intended to be limiting in describing the full scope of compositions and methods of this invention. Equivalent changes, modifications and variations of specific embodiments, materials, compositions and methods may be made within the scope of the present invention, with substantially similar results.

1. A method of treating a tissue defect in a human or other animal subject, comprising the steps of:
(a) culturing a living tissue in a medium to form a tissue culture;
(b) subjecting said tissue culture to an electromagnetic field;
(c) extracting said medium from said tissue culture; and
(d) administering said medium to the site of said tissue defect.

2. A method according to claim 1 wherein said tissue comprises endothelial cells.

3. A method according to claim 1 wherein said electromagnetic field stimulus is pulsed.

4. A method according to claim 1, wherein said tissue defect is in bone tissue.

5. A method according to claim 4, for the treatment of a defect associated with osteoporosis, spinal fixation procedure, joint replacement procedure, or bone fracture.

6. A method of enhancing cell proliferation in a tissue culture of interest, comprising the steps of:
(a) culturing a living tissue in a medium to form a first tissue culture;
(b) subjecting said first tissue culture to an electromagnetic field;
(c) extracting said medium from said first tissue culture; and
(d) administering said medium to said tissue culture of interest.

7. A method according to claim 6 wherein said electromagnetic field stimulus is pulsed.

8. A method according to claim 6 wherein said tissue comprises endothelial cells.

9. A composition for the treatment of tissue defects in a human or other animal subject, comprising a safe and effective amount of a medium produced by electromagnetic stimulation of a tissue culture.

10. A composition according to claim 9, wherein said tissue culture comprises endothelial cells.

11. A composition according to claim 9, additionally comprising a pharmaceutically-acceptable carrier.

12. A composition according to claim 11, wherein said carrier is selected from the group consisting of saline, hyaluronic acid, cellulose ethers (such as carboxymethyl cellulose), collagen, gelatin, an osteoconductive carrier, and mixtures thereof.

13. A composition according to claim 12, wherein said carrier comprises an osteoconductive carrier selected from the group consisting of bone particles, demineralized bone matrix, calcium phosphate, calcium sulfate, hydroxyapatite, polylactic acid, polyglycolic acid and mixtures thereof.

14. A composition according to claim 9, additionally comprising a growth active material selected from the group consisting of growth factors, hormones, phosphonates and mixtures thereof.
